(19) United States
(12) Patent Application Publication
(10) Pub. No.: US 2003/0083682 A1
(43) Pub. Date: May 1, 2003

(54) Ultrasonic revasculizer

(76) Inventor: Sean R. Heise, St Petersburg, FL (US)

Correspondence Address: DENNIS L. THOMTE, THOMTE, MAZOUR & NIEBERGALL, L.L.C., 2120 S. 72ND STREET, SUITE 1111, OMAHA, NE, 68124, US

(21) Appl. No.: 10/029,763

(22) Filed: Oct. 29, 2001

(51) Int. Cl. A61B 17/32

(52) U.S. Cl. 606/169

(57) ABSTRACT

An ultrasonic revasculizer for creating holes or openings in the heart to revasculize infarcted or blocked areas to create collateral blood flow to the damaged area of the heart is described which includes an elongated handle having an elongated flexible tubular neck extending from one end thereof. An enlarged depth guard is provided at the distal end of the neck and has an ultrasonic needle selectively longitudinally movably extending therethrough. A manual control in the form of a slide button is movably mounted in the handle and is operatively connected to the needle for controlling the longitudinal movement of the needle with respect to the depth guard. A depth gauge is provided on the handle adjacent the slide button which indicates the longitudinal position of the needle with respect to the depth guard. The needle is operatively connected to a source of ultrasonic power for ultrasonically driving the needle. In the revasculizer of this invention, the depth of the hole or opening in the heart is easily controllable.

### BACKGROUND OF THE INVENTION

1. Field of the Invention

This invention relates to an ultrasonic revasculizer for creating openings in the heart to revasculize infarcted or blocked areas to create collateral blood flow to the damaged area of the heart.

2. Description of the Related Art

It is frequently necessary to create openings in a human heart to enable revasculize infarcted or blocked areas to create collateral blood flow to the damaged area of the heart. In the past, the openings were created in the heart by means of a laser. Although the laser technique does create openings in the heart to revasculize infarcted or blocked areas to create collateral blood flow to the damaged area of the heart, the depth of the opening created by the laser is difficult to control.

### SUMMARY OF THE INVENTION

An ultrasonic revasculizer for creating holes or openings in the heart to revasculize infarcted or blocked areas to create collateral blood flow to the damaged area of the heart is described and includes an elongated handle having first and second ends. An elongated flexible tubular neck has its proximal end secured to the handle and has its distal end positioned remotely from the handle. An enlarged depth guard in the form of a disc is mounted on the distal end of the neck. An ultrasonic needle is selectively longitudinally movably mounted in the neck and extends from the neck and depth guard. A manual control is movably mounted on the handle which is operatively connected to the needle for controlling the selective longitudinal movement of the needle with respect to the depth guard. A depth gauge is provided on the handle adjacent the manual control which indicates the longitudinal position of the needle with respect to the depth guard. The needle is operatively connected to a source of ultrasonic power for ultrasonically driving the needle.

It is a principal object of this invention to provide an improved apparatus for creating holes or openings in the heart to revasculize infarcted or blocked areas to create collateral blood flow to the damaged area of the heart.

A further object of the invention is to provide an ultrasonic revasculizer for creating holes or openings in the heart to revasculize infarcted or blocked areas to create collateral blood flow to the damaged area of the heart.

Yet another object of the invention is to provide an ultrasonic revasculizer which includes an ultrasonic needle selectively movably extending from a depth guard.

Still another object of the invention is to provide an ultrasonic revasculizer having an ultrasonic needle thereon with the revasculizer including means for controlling the depth of penetration of the ultrasonic needle.

Yet another object of the invention is to provide an ultrasonic revasculizer for creating holes or openings in the heart to revasculize infarcted or blocked areas to create collateral blood flow to the damaged area of the heart which is relatively economical of manufacture, durable in use, and refined in appearance.

These and other objects will be apparent to those skilled in the art.

### BRIEF DESCRIPTION OF THE DRAWINGS

FIG. 1 is a perspective view of the ultrasonic revasculizer of this invention with the broken lines indicating alternate positions of the flexible neck;

FIG. 2 is a side view of the ultrasonic revasculizer of this invention which illustrates the depth guard positioned adjacent the human heart with the heart being indicated in broken lines; and

FIG. 3 is a view illustrating the revasculizer of this invention being used to create openings or holes in a human heart.

### DESCRIPTION OF THE PREFERRED EMBODIMENT

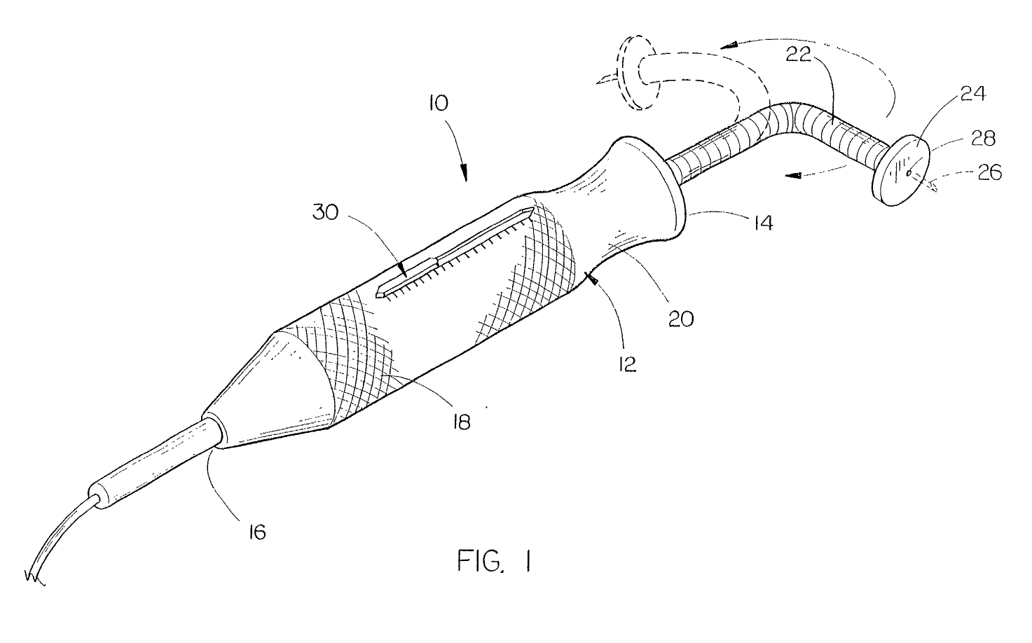

The ultrasonic revasculizer of this invention is generally indicated by the reference numeral 10 and includes an elongated handle having ends 14 and 16. The exterior surface of the handle 12 is provided with a textured surface generally indicated by the reference numeral 18 to enable the surgeon to conveniently grasp the instrument. As seen in FIG. 1, handle 12 includes a recessed portion 20 adjacent end 14 which aids the surgeon in gripping the instrument.

An elongated flexible tubular neck 22 extends from end 14 and has a disc-shaped depth guard 24 mounted on the distal end thereof. An ultrasonic needle 26 selectively movably extends from the depth guard 24 by way of the opening 28 formed in depth guard 24.

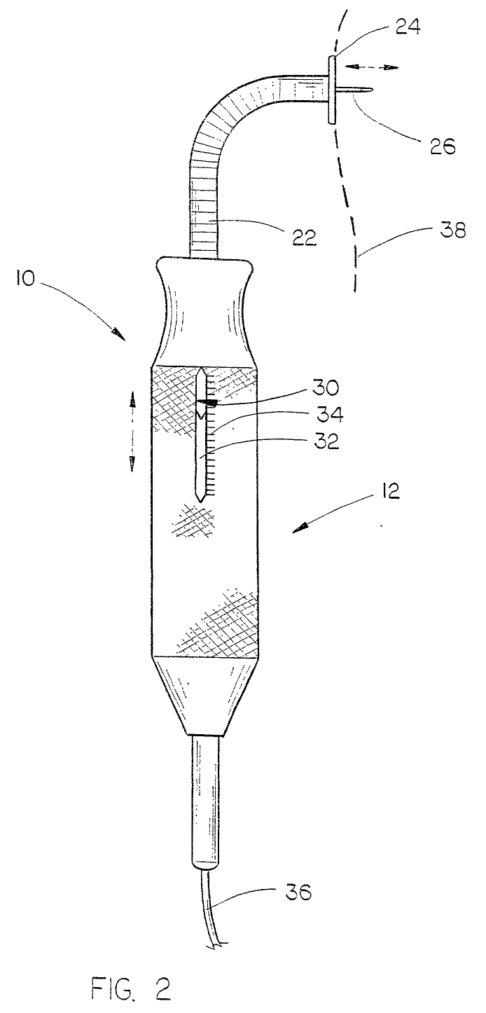

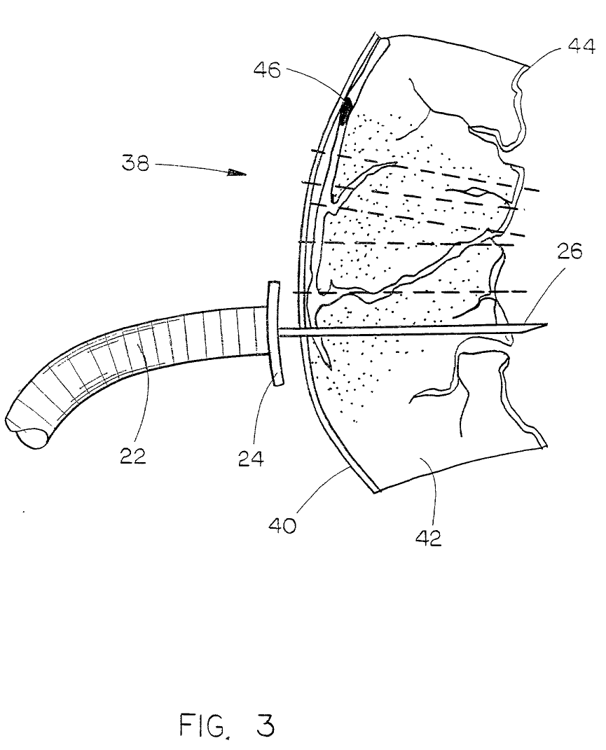

The numeral 30 refers to a manual control in the form of a slide button which is selectively movably mounted in slot or opening 32 formed in handle 12. The slide button 30 is connected to needle 26 by any convenient means such as a flexible wire, cable, etc. A depth gauge in the form of indicia 34 is provided on the exterior surface of the handle 12 adjacent the slot 32 for indicating the relative position of the needle 26 with respect to the depth guard 24. As seen in FIG. 2, when slide button 30 is in its uppermost position, the needle 26 is fully extended from the depth guard 24. When the slide button 30 is in its lowermost position in the slot 32, as illustrated in FIG. 1, the needle 26 is completely withdrawn into the neck 22. The needle 26 is operatively connected to a conventional source of ultrasonic power by means of the cable 36 extending from handle 12. In FIG. 2, the numeral 38 refers to the exterior surface of a human heart. In FIG. 3, the heart 38 is seen to include Epicardium 40, Myocardium 42 and Endocardium 44. The numeral 46 generally refers to a lesion or blockage in the Epicardial artery.

In operation, when it is desired to create openings or holes in the heart to revasculize infarcted or blocked areas to create collateral blood flow to the damaged area of the heart, the patient's chest is normally opened and the neck 22 is inserted through the chest opening. The instrument is manipulated so that the depth guard 24 is positioned adjacent the exterior surface of the patient's heart, as illustrated in FIG. 3. The slide button 30 would initially be preferably positioned in the position as illustrated in FIG. 1 so that the needle 26 does not extend from depth guard 24. The surgeon then actuates the source of ultrasonic power to activate ultrasonic needle 26. When the needle 26 has been activated, the slide button 30 is moved upwardly in the slot 32 until the needle 26 has penetrated the heart a predetermined amount, as indicated by the depth gauge 30.

The above-described procedure is repeated until the required number of openings or holes have been created in the heart to revasculize infarcted or blocked areas to create collateral blood flow to the damaged area of the heart.

It can therefore be seen that the use of an ultrasonic needle wherein the depth of penetration of the needle in the heart is controlled, thereby overcoming the problem with the prior art methods of creating holes or openings in the heart wherein the depth of the same was not controlled.

Thus it can be seen that the invention accomplishes at least all of its stated objectives.

I claim:

1. An ultrasonic revasculizer for creating holes or openings in the heart to revasculize infarcted or blocked areas to create collateral blood flow to the damaged area of the heart, comprising:
an elongated handle having first and second ends;
an elongated tubular neck having proximal and distal ends;
said proximal end of said neck being secured to said handle at said first end thereof and extending therefrom;
an enlarged depth guard at said distal end of said neck;
an ultrasonic needle selectively longitudinally movably extending from said neck and said depth guard;
a manual control movably mounted on said handle which is operatively connected to said needle for controlling the longitudinal movement of said needle with respect to said depth guard;
a depth gauge on said handle which indicates the longitudinal position of said needle with respect to said depth guard;
said needle being operatively connected to a source of ultrasonic power for ultrasonically driving said needle.

2. The revasculizer of claim 1 wherein said neck is flexible.

3. The revasculizer of claim 1 wherein said manual control comprises a slide button slidably movably mounted on said handle.

4. The revasculizer of claim 1 wherein said depth guard comprises a flat, disc-shaped member positioned on said distal end of said neck.

5. The revasculizer of claim 3 wherein said depth gauge comprises indicia marks positioned on said handle adjacent said slide button.
